(19) United States
(12) Patent Application Publication
(10) Pub. No.: US 2012/0245197 A1
(43) Pub. Date: Sep. 27, 2012

(54) PHARMACEUTICAL COMPOSITION CONTAINING TOSUFLOXACIN OR SALT THEREOF

(75) Inventor: Setsuko NISHIMURA, Toyama (JP)

(73) Assignee: Toyama Chemical Co., Ltd., Shinjuku-ku (JP)

(21) Appl. No.: 13/053,515

(22) Filed: Mar. 22, 2011

(51) Int. Cl. A61K 31/4375; A61P 31/04

(52) U.S. Cl. 514/300

(57) ABSTRACT

A pharmaceutical composition containing tosufloxacin or a salt thereof, which is excellent in stability and preservative effect and contamination preventing effect is provided. A pharmaceutical composition containing tosufloxacin or a salt thereof, a metal compound comprising aluminum, borax, an alkali metal chloride and a preservative is excellent in stability and preservative effect and contamination preventing effect, and useful especially as eye drops, nasal drops and ear drops.

### BACKGROUND OF THE INVENTION

(1) Field of the Invention

The present invention relates to a pharmaceutical composition containing tosufloxacin (7-(3 -amino-1-pyrrolidinyl)-1-(2,4-difluorophenyl)-6-fluoro-1,4-dihydro-4-oxo-1,8-naphthyridine-3-carboxylic acid) or a salt thereof which exhibits antimicrobial activity, a metal compound comprising aluminum, borax, an alkali metal chloride and a preservative, which is excellent in stability and safety.

(2) Description of Related Art

Tosufloxacin is well known as a quite excellent antimicrobial agent against not only gram-positive bacteria but also gram-negative bacteria. Moreover, as a method to stabilize its solution, a method to add a metal compound comprising aluminum, borax, and an alkali metal chloride [Japanese Patent No. 3346586] is known.

By the way, an aqueous liquid formulation which is applied for eye drops, nasal drops, ear drops and the like is often used little by little over a long period. As a result, the aqueous liquid formulation is in danger of contamination.

So far, a pharmaceutical composition containing tosufloxacin or a salt thereof, a metal compound comprising aluminum, borax, an alkali metal chloride and a preservative has not been known.

A pharmaceutical composition containing tosufloxacin or a salt thereof, which has preservative effect and contamination preventing effect is desired.

### BRIEF SUMMARY OF THE INVENTION

In such a situation, the present inventor, having earnestly studied, has found that a pharmaceutical composition containing tosufloxacin or a salt thereof, a metal compound comprising aluminum, borax, an alkali metal chloride and a preservative is excellent in stability and preservative effect and contamination preventing effect, and has accomplished the present invention.

The pharmaceutical composition of the present invention, containing tosufloxacin or a salt thereof, a metal compound comprising aluminum, borax, an alkali metal chloride and a preservative, is excellent in stability and preservative effect and contamination preventing effect, and useful especially as eye drops, nasal drops and ear drops.

### DETAILED DESCRIPTION OF THE INVENTION

The present invention will be described in detail below.

The pharmaceutical composition of the present invention may be produced by adding a metal compound comprising aluminum to a suspension of tosufloxacin or a salt thereof and dissolving them, followed by adjusting pH with addition of borax, adding an alkali metal chloride to the solution for isotonization, and adding a preservative and dissolving it in the solution.

The salt of tosufloxacin used in the pharmaceutical composition of the present invention includes: for instance, salts with inorganic acid such as hydrochloric acid, sulfuric acid and phosphoric acid; salts with organic acid such as acetic acid, lactic acid, succinic acid, p-toluenesulfonic acid, methanesulfonic acid, maleic acid, malonic acid and gluconic acid; salts with an amino acid such as aspartic acid and glutamic acid; sodium salt and potassium salt, and salt with p-toluenesulfonic acid is preferred.

Moreover, when solvates, hydrates and various forms of crystals are present in tosufloxacin or a salt thereof, the present invention may use all of them and preferably use a hydrate of tosufloxacin p-toluenesulfonic acid salt.

The amount of tosufloxacin or a salt thereof used in the pharmaceutical composition is 0.1 to 3% and preferably 0.3%.

The alkali metal chloride used in the pharmaceutical composition of the present invention includes, for instance, sodium chloride and potassium chloride, and sodium chloride is preferred.

The amount of the alkali metal chloride used is that required for isotonization of the solution.

The metal compound comprising aluminum used in the pharmaceutical composition of the present invention includes, for instance, aluminum potassium sulfate, aluminum chloride and aluminum sulfate and hydrates thereof, and aluminum potassium sulfate 12-hydrate is preferred.

The amount of the metal compound comprising aluminum used is usually 0.1 to 30-fold moles and preferably 0.1 to 10-fold moles to that of tosufloxacin or a salt thereof.

The pH of the pharmaceutical composition of the present invention is 3.5 to 7.0 and preferably 4.0 to 6.5 during dissolution, and the pH is adjusted by the amount of borax used. If needed, other alkalis or acids may be added to the pharmaceutical composition of the present invention. The alkalis include, for instance, sodium hydroxide, potassium hydroxide, sodium carbonate and potassium carbonate. The acids include, for instance, hydrochloric acid, sulfuric acid, phosphoric acid, acetic acid, p-toluenesulfonic acid and methanesulfonic acid.

The preservative used in the pharmaceutical composition of the present invention includes benzethonium chloride, methyl parahydroxybenzoate, ethyl parahydroxybenzoate, chlorobutanol, sodium dehydroacetate and sodium benzoate, and ethyl parahydroxybenzoate is preferred.

The amount of the preservative used in the pharmaceutical composition is preferably 0.001 to 0.1% and more preferably 0.03 to 0.05%.

An example of the preferred pharmaceutical composition of the present invention is a liquid formulation obtained by suspending a hydrate of tosufloxacin p-toluenesulfonic acid salt in water, adding aluminum potassium sulfate 12-hydrate to the suspension and dissolving them, followed by adjusting pH with addition of borax, adding sodium chloride to the solution for isotonization, and further adding a preservative.

The pharmaceutical composition of the present invention is applicable to formulations mainly for injections (solution or dissolved-before using type), for eye drops, for nasal drops, for ear drops, for oral cavity or for oral administration. In preparing the formulations, pharmaceutically acceptable additives may be further added. The additives include diluents, stabilizers, buffers, antioxidants, taste masking agents, thickening agents and soothing agents.

The method of the administration, dosage, and dosage frequency may be appropriately selected according to the age, the weight and the symptoms of patients. The pharmaceutical composition of the present invention may be usually administered to adults orally or parenterally (for example, injection or administration into the rectum) in a dosage of 0.1 to 50 mg/kg/day in terms of tosufloxacin at once or in several divided doses. In case of administrations such as eye drops, ear drops and nasal drops, the pharmaceutical composition of the present invention may be administered in a dosage of 1 to 100 pg/kg/day in terms of tosufloxacin at once or in several divided doses.

### EXAMPLE

Next, the present invention will be described with examples, a comparative example and test examples, but the present invention is not limited to these examples.

As compound A, a hydrate of tosufloxacin p-toluenesulfonic acid salt was used.

The comparative formulation in Test Example 2 is an eye drop produced without adding a preservative in the same way as Example 1.

### Example 1

0.3 g of compound A and 0.12 g of aluminum potassium sulfate 12-hydrate were added to 90 mL of purified water and dissolved at 55° C. 0.1 g of borax and 0.1 g of ethyl parahydroxybenzoate were added to the solution and dissolved. The solution was cooled to room temperature and isotonized with sodium chloride. It was adjusted to pH 5.2 with sodium hydroxide solution, and diluted to 100 ml of total volume with purified water. The solution was sterile-filtered through a membrane filter (0.22 μm) and then filled into an eye drop bottle made of polyethylene to produce an eye drop.

### Example 2

Using 0.05 g of ethyl parahydroxybenzoate, an eye drop was produced in the same way as Example 1.

### Example 3

Using 0.03 g of ethyl parahydroxybenzoate, an eye drop was produced in the same way as Example 1.

### Example 4

Using 0.02 g of ethyl parahydroxybenzoate, an eye drop was produced in the same way as Example 1.

### Comparative Example 1

0.3 g of compound A and 0.12 g of aluminum potassium sulfate 12-hydrate were suspended in 90 mL of purified water and dissolved at 55° C. 0.1 g of borax and 0.82 g of sodium chloride were dissolved in the solution. Moreover, 7 ml of purified water and 3 ml of 1 mg/ml aqueous benzalkonium chloride solution were added to the resulting solution. An insoluble matter was observed after addition of benzalkonium chloride.

### Test Example 1 (Effect of the Preservative On the Stability of the Formulation)

The formulation of Example 2 was stored at room temperature and at refrigerated temperature, and after 6 weeks, the concentration of compound A was measured.

The residual rate of compound A after 6 weeks was calculated. residual rate (%)=(concentration of compound A after 6 weeks/concentration of compound A at initiation time)×100

The result is shown in Table 1.

TABLE 1

| storage conditions | residual rate (%) |
|---|---|
| room temperature | 99.8 |
| refrigerated temperature | 99.9 |

The formulation of Example 2 was stable.

### Test Example 2 (Preservative Effect)

The preservative effect was determined using the broth microdilution method according to the reference method for broth dilution antifungal susceptibility testing of yeasts M27-A3 and the reference method for broth dilution antifungal susceptibility testing of filamentous fungi M38-A2 recommended by Clinical and Laboratory Standards Institute (CLSI). Sterile-filtered 0.67% Difco Yeast Nitrogen Base without Amino Acids (YNB) added with 0.5% glucose was used as the medium for susceptibility testing.

Fungal inoculum of Candida albicans TIMM1623 were prepared by diluting cryopreserved fungal suspension with YNB having 10 times higher concentration so that the concentration of the fungal inoculum would be 10 times higher than that of final fungal inoculum (1×10³ CFU/mL).

Fungal inoculum of Aspergillus fumigatus TIMM0063 were prepared by diluting refrigerated fungal suspension with YNB having 10 times higher concentration so that the concentration of the fungal inoculum would be 10 times higher than that of final fungal inoculum (1×10⁴ CFU/mL).

180 μL of examples 1 to 4 and the comparative formulation were diluted with distilled water in 10 consecutive twofold dilutions on a multi well plate MS-8096R for suspension culture (SUMITOMO BAKELITE CO., LTD., 96-well, flat bottom). Then, the fungal inocula were inoculated into each well by 20 μL and suspended with a microplate mixer. After cultivating at 35° C. for 48 hours, preservative effects were determined by visual inspection. In the case of yeasts, culture solutions were suspended using a microplate mixer prior to determination.

The maximum dilution ratios in which optically clear wells were observed were determined in order to use them as indices of the preservative effects. The result is shown in Table 2.

TABLE 2

maximum dilution ratios in which growth
inhibitions were observed

| | C. albicans | A. fumigatus |
|---|---|---|
| test substance | TIMM1623 | TIMM0063 |
| Example 1 | 2 | 2 |
| Example 2 | 1 | 2 |
| Example 3 | 1 | 1 |
| Example 4 | —^{a} | 1 |
| Comparative | —^{b} | —^{b} |
| formulation | | |

^{a}Not less than 50% of growth inhibition was observed in a stock solution.
^{b}Growth inhibitions were not observed.

In the formulation which was not added with ethyl parahydroxybenzoate, a preservative, growth inhibitions of fungi were not observed. However, in the formulation containing 0.03 to 0.1% ethyl parahydroxybenzoate, growth of fungi was completely inhibited.

As is apparent from the above results, the pharmaceutical composition of the present invention containing tosufloxacin or a salt thereof, a metal compound comprising aluminum, borax, an alkali metal chloride and a preservative had stability and preservative effect and contamination preventing effect, and was quite excellent especially as eye drops, nasal drops and ear drops.

1. A pharmaceutical composition comprising 7-(3-amino-1-pyrrolidinyl)-1-(2,4-difluorophenyl)-6-fluoro-1,4-dihydro-4-oxo-1,8-naphthyridine-3-carboxylic acid or a salt thereof, a metal compound comprising aluminum, borax, an alkali metal chloride and a preservative.

2. The pharmaceutical composition according to claim 1, wherein the preservative is ethyl parahydroxybenzoate.

3. The pharmaceutical composition according to claim 1, wherein the metal compound comprising aluminum is aluminum potassium sulfate 12-hydrate, the alkali metal chloride is sodium chloride and the preservative is ethyl parahydroxybenzoate.

4. The pharmaceutical composition according to claim 3, wherein the content of the ethyl parahydroxybenzoate in the pharmaceutical composition is 0.001 to 0.1%.
